(19) United States
(12) Patent Application Publication
(10) Pub. No.: US 2008/0146809 A1
(43) Pub. Date: Jun. 19, 2008

(54) Process for the Preparation of Novel Amorphous Montelukast Sodium

(75) Inventors: Chava Satyanarayana, Secunderabad (IN); Gorantla Seeta Ramanjaneyulu, Secunderabad (IN); Indukuri Venkata Sunil Kumar, Hyderabad (IN)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Matrix Laboratories Ltd, Secunderabad (IN)

(21) Appl. No.: 11/794,277

(22) PCT Filed: Nov. 11, 2005
(86) PCT No.: PCT/IN05/00366

§ 371 Date: Jun. 22, 2007

(30) Foreign Application Priority Data
Nov. 19, 2004 (IN) 1221/CHE/2004

(51) Int. Cl. C07D 215/12

(52) U.S. Cl. 546/174

(57) ABSTRACT

A process for the preparation of novel amorphous montelukast sodium by dissolving montelukast free acid in an organic solvent, converting into its alkali salt followed by vacuum drying or spray drying the solution. Alternatively by dissolution of montelukast sodium in organic solvent followed by vacuum drying or spray drying the solution.

### BACKGROUND OF THE INVENTION

Montelukast sodium namely Sodium 1-[[[(1R)-1-[3-[(1E)-2-(7-chloro-2-quinolinyl)ethenyl]phenyl]-3-[2-(1-hydroxy-1-methylethyl)phenyl]propyl]thio]methyl]cyclo propane acetic acid has the formula

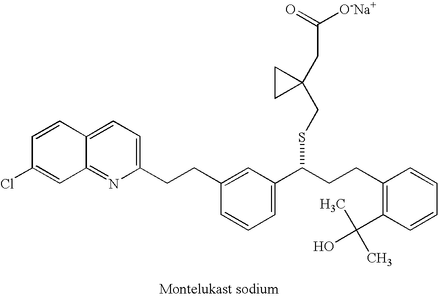

Montelukast sodium is a leukotriene antagonist and inhibits the synthesis of leukotriene biosynthesis. It is useful as anti-asthmatic, anti-allergic, anti-inflammatory, cytoprotective agent and hence useful in the treatment of angina, cerebral spasm, glomerular nephritis, hepatic, end toxemia, uveitis and allograft rejection.

European Patent No. 480,717 discloses montelukast sodium along with other related compounds and methods for their preparation. The reported method of synthesis proceeded through corresponding methyl ester namely, Methyl 2-[(3S)-[3-[(2E)-(7-chloroquinolin-2-yl)ethenyl]phenyl]-3-hydroxypropyl]benzoate and involves coupling methyl 1-(mercaptomethyl)cyclopropane acetate with a mesylate generated in-situ. The methyl ester of montelukast is hydrolyzed to free acids and the latter converted directly to montelukast sodium salt. The process is not particularly suitable for large-scale production because it requires tedious chromatographic purification of the methyl ester intermediate and /or the final product with low yields.

U.S. Pat. No. 5,614,632 discloses that the products obtained as per EP 480,717 are amorphous sodium salts, which are hydrated and often not ideal for pharmaceutical formulation and therefore provides crystalline montelukast sodium. It further discloses the process for preparation of crystalline montelukast sodium, which comprises
Treating crystalline polymorphic forms (Form-A, Form-B) of montelukast dicyclohexyl amine salt with acetic acid in mixture of toluene and water,
Separating the organic layer
Treating the organic layer containing free acid with sodium hydroxide in 1% aqueous ethanol,
Evaporating the solvent and crystallizing montelukast sodium from mixture of toluene and acetonitrile.

PCT publication WO 03/066598 discloses the anhydrous amorphous form of montelukast sodium and the process for its preparation. The disclosed process involves the dissolution of montelukast free acid in aromatic hydrocarbon solvent or halogenated solvent and converting the free acid to its sodium salt, accompanied by addition of a cyclic or acyclic hydrocarbon solvent or mixtures thereof, followed by isolation and drying the material for compound.

The disclosed prior art processes involves the crystallization of montelukast sodium either in amorphous or crystalline form from solution. During crystallization or isolation from solution, some quantity of product may loose due to the solubility of the product thereby increase the product cost.

The inventors found a process by which the product loss during isolation can be minimized by spray drying/vacuum drying of the solution.

### SUMMARY OF THE INVENTION

The main object of the present invention is to provide a novel amorphous forms of montelukast and its alkali salts.

Another object of the invention is to provide a process for the preparation of amorphous forms montelukast and its alkali salts

Another object of the present invention is to provide a process for the preparation of amorphous form of montelukast sodium.

Yet another object of the present invention is to provide a process for the preparation of montelukast and its alkali salts by dissolution of montelukast free acid in organic solvent, converting into its alkali salt followed by vacuum drying or spray drying of the solution.

Yet another object of the present invention is to provide the process of for preparation of novel amorphous montelukast sodium by dissolution of montelukast sodium in organic solvent followed by vacuum drying or spray drying of the solution.

The solution of montelukast free acid obtained by either dissolution of solid in organic solvent(s) or in solution form obtained by neutralization of montelukast base salts is treated with molar equivalents of sodium hydroxide solution, removing insolubles (if any), followed by spray drying or vacuum drying the solution affords the novel amorphous montelukast sodium.

In another embodiment of the present invention in which montelukast sodium of any forms is dissolved in organic solvent(s) followed by vacuum drying or spray drying affords the novel amorphous montelukast sodium.

### BRIEF DESCRIPTION OF THE DRAWINGS

FIG. 1: X-Ray diffraction pattern of the amorphous montelukast sodium of the present invention

FIG. 2: X-Ray diffraction pattern of the reported amorphous montelukast sodium (WO 03/066598)

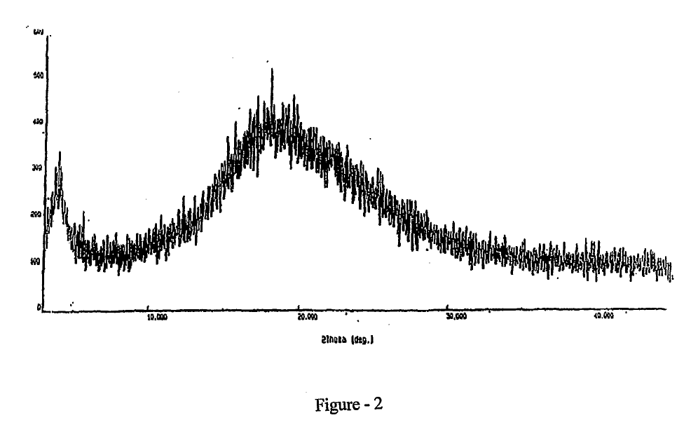

### DETAILED DESCRIPTION OF THE INVENTION

The process of the present invention comprises:
Dissolving montelukast free acid in organic solvent(s)
Treating with molar equivalents of sodium hydroxide solution
Removing insolubles (if any)
Drying the solution by vacuum drying or spray drying

### Optionally

Dissolving montelukast sodium in organic solvent(s)
Removing insolubles if any
Drying the solution by vacuum drying or spray drying

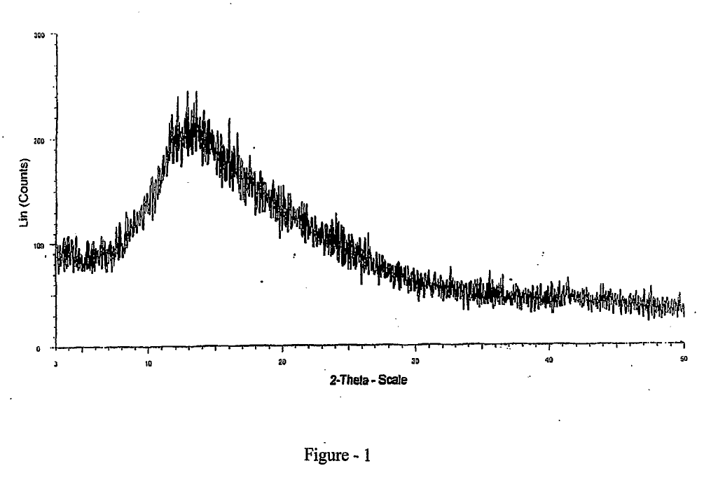

The prepared montelukast sodium is a novel amorphous form and exhibits a typical XRD pattern as shown in FIG. 1.

The starting material of montelukast free acid or montelukast base salts are prepared by the literature reported methods.

Montelukast free acid is dissolved in organic solvent(s) like methanol, ethanol, isopropanol, toluene, aectonitrile, ethyl acetate, isopropyl acetate, acetone or mixture of above solvents is treated with sodium hydroxide solution in ethanol, insolubles are removed by filtration and the clear solution of montelukast sodium is dried under vacuum at temperature of 60° C. to 95° C. till constant weight.

The montelukast free acid in solution form obtained by neutralization of montelukast base salts can be used as such for the preparation of amorphous montelukast sodium without isolation of montelukast free acid in solid form.

Alternately the montelukast sodium solution obtained by dissolution of montelukast free acid in organic solvent(s) such as methanol, ethanol, isopropanol, toluene, aectonitrile, ethyl acetate, isopropyl acetate, acetone or mixture thereof is treated with sodium hydroxide solution in ethanol, insolubles are removed by filtration and the clear solution of montelukast sodium is spray dried at inlet temperature of 65° C. to 175° C. which has resulted the out let temperature of 50° C. to 120° C. under the flow of inert gases or nitrogen.

Optionally the solution of montelukast sodium obtained by dissolution of montelukast sodium in any known forms in organic solvent(s) such as methanol, ethanol, isopropanol, toluene, aectonitrile, ethyl acetate, isopropyl acetate, acetone or mixture thereof is spray dried at inlet temperature of 65° C. to 175° C. which has resulted the out let temperature of 50° C. to 120° C. under the flow of inert gases or nitrogen.

The invention is now illustrated with a few non-limiting examples.

### EXAMPLE I

### Preparation of Amorphous Montelukast Sodium

Montelukast free acid (70 g) is dissolved in methanol (210 ml), 0.486 molar solution of sodium hydroxide in ethanol (230 ml) is added, mixed for about 10-15 min, filtered the resultant reaction mass through hyflow bed, washed the bed with 35 ml methanol. The clear solution is spray dried with spray drier at inlet temperature of 110° C., outlet temperature of 73° C. to 75° C., nitrogen flow rate of 40-50 mm, with a solution feed rate of 30%.

Dry wt of the amorphous sodium is 62.0 g, (yield of 52.8%).

XRD is as shown in FIG. 1.

The various solvents used for dissolution of montelukast free acid and the parameters of Spray drier to obtain amorphous montelukast sodium is given in below (Table-1)

TABLE 1

| | | Inlet | Outlet | | Solution feed |
|---|---|---|---|---|---|
| | | Temp | temp | N₂ flow rate | rate |
| S. No | Solvent | (° C.) | (° C.) | (mm) | (%) |
| 1 | Ethanol | 110 | 75 | 40 | 30 |
| 2 | Toluene | 160 | 112 | 4 | 30 |
| 3 | Ethyl acetate | 120 | 75 | 45 | 35 |
| 4 | Acetone | 90 | 63 | 45 | 35 |
| 5 | Toluene- | 110 | 81 | 40 | 30 |
| | Acetonitrile | | | | |
| 6 | Toluene- | 170 | 114 | 45 | 35 |
| | Acetonitrile | | | | |
| 7 | Isopropanol | 75 | 54 | 35 | 40 |
| 8 | Isopropyl acetate | 90 | 66 | 30 | 45 |
| 9 | Methanol-ethyl | 76 | 56 | 30 | 45 |
| | acetate | | | | |

### EXAMPLE II

### Preparation of Amorphous Montelukast Sodium by Spray Drying of Montelukast Sodium Solution

Montelukast sodium (100 g) is dissolved in methanol (200 ml) at 27° C. to 30° C. by mixing for about 15 min, filtered the solution to remove insolubles, clear solution is spray dried at the set of parameters inlet temperature of 90° C., outlet temperature of 75° C., Nitrogen flow rate of 45 mm, solution feed rate of 40%.

The dry weight of the amorphous montelukast sodium is 65 g (yield is 65%)

XRD is as shown in FIG. 1

1. A process for the preparation of amorphous montelukast sodium comprising
dissolving montelukast free acid in an organic solvent or a mixture of organic solvents,
treating with molar equivalents of sodium hydroxide solution,
removing insolubles (if any),
drying the solution by vacuum drying or spray drying,
which is characterized by the typical X-ray diffractions as shown in FIG. 1.

2. A process as claimed in claim 1, wherein the montelukast free acid is either in solid form or in solution form obtained from montelukast base salts.

3. A process as claimed in claim 1, wherein the organic solvent(s) is methanol, ethanol, isopropanol, toluene, aectonitrile, ethyl acetate, isopropyl acetate, acetone or a mixture thereof.

4. A process for the preparation of amorphous montelukast sodium comprising
dissolving montelukast sodium in an organic solvent or a mixture of organic solvents,
removing the insolubles (if any),
removing the solvent either by vacuum drying or by spray drying,
which is characterized by the typical X-ray diffractions as shown in FIG. 1.

5. A process as claimed in claim 1, wherein the organic solvent(s) is methanol, ethanol, isopropanol, toluene, aectonitrile, ethyl acetate, isopropyl acetate, acetone or a mixture thereof.

6. A process as claimed in claim 4, wherein the utilized montelukast sodium is in a known amorphous or crystalline forms.

7. Novel amorphous montelukast sodium characterized by the XRD pattern as shown in FIG. 1.
